(19) United States
(12) Patent Application Publication
(10) Pub. No.: US 2025/0375206 A1
(43) Pub. Date: Dec. 11, 2025

(54) User-Friendly Junctional Tourniquet Device

(71) Applicant: InnoVital Systems, Inc., Calverton, MD (US)

(72) Inventors: Curt Steven Kothera, Laurel, MD (US); Amit Navin Shah, North Potomac, MD (US)

(73) Assignee: InnoVital Systems, Inc., Calverton, MD (US)

(21) Appl. No.: 19/233,633

(22) Filed: Jun. 10, 2025

Related U.S. Application Data
(60) Provisional application No. 63/658,093, filed on Jun. 10, 2024.

(51) Int. Cl. A61B 17/132; A61B 17/00; A61B 90/00

(52) U.S. Cl. CPC A61B 17/1325; A61B 2017/00407; A61B 2090/064; A61B 2090/0807

(57) ABSTRACT

The present invention relates to a device that is placed for inguinal or axillary application on a patient that is suffering from junctional hemorrhage to occlude the vessels and stop bleeding in a manner that overcomes many shortcomings of existing designs and related devices.

### CROSS-REFERENCE TO RELATED APPLICATIONS

This application claims the benefit of priority of U.S. Provisional Pat. App. Ser. No. 63/658,093, filed Jun. 10, 2024, the contents of which are hereby incorporated by reference in their entirety.

### STATEMENT REGARDING FEDERALLY SPONSORED RESEARCH OR DEVELOPMENT

The invention disclosed herein was conceived and reduced to practice without federal funding.

### BACKGROUND OF THE INVENTION

The present invention relates to a user-friendly device for stopping junctional hemorrhage in a patient through blood vessel occlusion.

Hemorrhage is a leading cause of preventable death on the battlefield [Eastridge, B. J., Mabry, R. L., et al., Death on the battlefield (2001-2011): Implications for the future of combat casualty care, J. Trauma Acute Care Surg., 2012 73(6): Supplement 5: S431-S437; van Oostendorp, S. E., Tan, E. C. T. H., Geeraedts, L. M. G., Prehospital control of life-threatening truncal and junctional hemorrhage is the ultimate challenge in optimizing trauma care; a review of treatment options and their applicability in the civilian trauma setting, Emergency Medicine, 2016 24:110 (13 pp.)] and in civilian trauma patients [Teixeira, P. G. R., Inaba, K., et al., Preventable or Potentially Preventable Mortality at a Mature Trauma Center, J. Trauma: Injury, Infection, and Critical Care, 2007 63(6): 1338-1347; Tien, H. C., Spencer, F., Tremblay, L., et al., Preventable Deaths from Hemorrhage at a Level I Canadian Trauma Center, J. Trauma: Injury, Infection, and Critical Care, 2007 62(1): 142-146].

Multiple studies have shown that prompt tourniquet use can decrease both military and civilian deaths. However, there is significant evidence that laypeople generally fail to apply conventional extremity tourniquets (e.g., combat application tourniquet or “CAT”) correctly. The success rate among lay users applying the CAT is about 20% when no instructions are provided [Goolsby, C., Branting A., et al., Just-in-Time to Save Lives: A Study of Layperson Tourniquet Application, Academic Emergency Medicine September 2015 22(9): P.1113-7] and about 50% when just-in-time instructions are provided [Goolsby, C., Chen, E., Branting, A., Weissbrod, E., David, J., Moore, K., Olsen, C., Analysis of Layperson Tourniquet Application Using a Novel Color-Coded Device, Disaster Medicine and Public Health Preparedness April 2016 Vol. 10(2) P. 274-280].

Moreover, there is significant evidence of rapid degradation of tourniquet application skills among both trained laypeople and trained combat medics [Goralnick E, Chaudhary M A, McCarty J C, et al., Effectiveness of Instructional Interventions for Hemorrhage Control Readiness for Laypersons in the Public Access and Tourniquet Training Study (PATTS): A Randomized Clinical Trial. JAMA Surg. Sep. 1, 2018 153(9):791-799; Landman A, de Vries D, Binsch O., Retention of military combat lifesaving skills during six months following classroom-style and individualized-style initial training, Mil. Psychol. November-December 2023 35(6):590-602].

Given the higher complexity of junctional tourniquets when compared to extremity tourniquets, frequent application failure is to be expected among minimally trained and untrained personnel. Multiple studies detail common failure points with existing junctional tourniquets as applied by trained users including long assembly and application times (with ongoing blood loss); inadequate pre-tensioning; and failure during transport [Kheirabadi B S, Terrazas I B, Hanson M A, Kragh J F J, et al., In vivo assessment of the Combat Ready Clamp to control junctional hemorrhage in swine, J. Trauma Acute Care Surg. 2013 74:1260-5; Kotwal R S, Butler F K, Gross K R, Kheirabadi B S, Baer D G, et al., Management of junctional hemorrhage in tactical combat casualty care: TCCC guidelines proposed change 13-03, J. Spec. Oper. Med. 2013 13:85-93; Theodoridis, C. A., Kafka, K. E., Perez, A. M., et al., Evaluation and Testing of Junctional Tourniquets by Special Operation Forces Personnel: A Comparison of the Combat Ready Clamp and the Junctional Emergency Treatment Tool, J. Spec. Oper. Med. 2016 16(1):44-50; Sulava E., Thompson C., Lesko J., et al., Axillary Use of Three Junctional Tourniquet Devices in Human Volunteers, Ann. Emergency Medicine 2022 Volume 80, Issue 4, Supplement, Page S117]. These failure modes are expected to be magnified when junctional tourniquets are applied by minimally trained personnel.

The present invention builds upon the existing state of the art for junctional tourniquets and adds features that aid the operator in obtaining correct positioning, ease and speed of use, and correct use.

### SUMMARY OF THE INVENTION

The user-friendly junctional tourniquet of the present invention is an easy-to-use device that is designed to facilitate correct use. It is designed to help overcome many of the existing problems encountered during use of existing devices, including: incorrect position on patient, by offering clear graphical instructions included with and on the device; incorrect tightness (too loose), by offering a needle gauge that is visible to the user to see real-time feedback of tightness and a biased tightening knob that reduces the likelihood of turning it in the wrong (loosening) direction; incomplete application procedure, by offering clear graphical instructions, color-coding, part numbers, steps, and using common symbols; difficult device assembly, by offering a pre-assembled device; difficult device use or slow application and interruption of pressure delivery during application, by offering high usability features and designing for rapid transition from manual pressure delivery to device-delivered pressure; decrease in tourniquet pressure after application, by offering a needle gauge that is visible to the user or other bystanders showing current pressure level; and loosening (e.g., during transit), by offering a screw mechanism with high friction to prevent unwinding.

A user-friendly junctional tourniquet according to the present disclosure will have at least the following features to help guide unskilled users in correctly applying the device to a patient in need:

It will include compact stowage so the device may be removed from packaging as a single unit without dangling parts and loose straps that can get tangled and overwhelm the lay user, especially in scenarios with high situational duress. All parts are ideally preassembled on the device. Common snap-fit features will hold parts together and release in a known manner. A strap is initially wound up in a component that resembles a seat belt reel, making it familiar to users.

Usage guidance incorporating redundant modalities to facilitate correct application by unskilled users will be included. Sequential numbering of key procedure steps will be employed on the device parts relevant for each step. Color-coding of parts and commonly understood symbols will accompany the numbering as additional visual cues to guide the user to complete the steps correctly. Instructional graphics (for example, stickers) will also be placed visibly on the top side of the device to provide additional means of real-time instructions.

Real-time feedback to the user will supplement the usage guidance features, guiding the user to complete all of the necessary steps in the correct sequence. Real-time feedback will be provided to the user in the form of an intuitive gauge that is contained in the tightening knob (e.g., similar to a tire pressure gauge). This will indicate to the user where they are in the application of critical pressure to control the bleeding in the patient and when they can stop tightening the knob (e.g., tuning the gauge for 300 mmHg).

Safety features will be included in the design, in addition to the aforementioned device features. Examples of safety features include a knob with offset prongs to make it much more difficult for a user to turn the knob in the wrong direction and waste critical time not applying pressure to the patient; and an over-pressure slipring at the connection between the knob and tightening shaft, so if too much torque is applied to the knob it will slip and not transfer to the patient. The direction the knob is to be turned will also be indicated by a curved arrow.

A preferred embodiment of the user-friendly junctional tourniquet of the present invention has four key steps in its application procedure and one additional monitoring step: (1) apply manual pressure to site via paddle (axillary or inguinal); (2) fasten strap around patient while continuing manual paddle pressure delivery; (3) remove slack from strap while continuing manual paddle pressure delivery; (4) position and turn knob to apply final pressure; and (5) track pressure applied and monitor for loosening, then tighten as needed.

These steps may vary somewhat depending on the features included in each particular embodiment of the invention. Below is a more detailed description of how these steps apply to the preferred embodiment detailed above as an example but is not intended to be limiting. One skilled in the art will readily understand how the steps may be similarly applied in alternative embodiments.

### BRIEF DESCRIPTION OF THE DRAWINGS

FIG. 1 illustrates a perspective view of an embodiment of a user-friendly junctional tourniquet device according to the present invention, in a stowed state.

FIG. 2 illustrates a perspective view of the embodiment of FIG. 1, with manual pressure pad removed.

FIG. 3 illustrates a preferred embodiment of the present invention in a deployed state.

FIG. 4 illustrates a primary pressure subassembly according to the present invention.

FIG. 5 is an exploded view of the parts of a primary pressure subassembly according to the present disclosure.

FIG. 6 illustrates a spool subassembly according to the present disclosure.

FIG. 7 is an exploded view of the parts of the spool subassembly of FIG. 6.

FIG. 8 illustrates a perspective view of the user side of a primary pressure subassembly according to the present disclosure, in the stowed configuration.

FIG. 9 depicts a perspective view of the user side of the primary pressure subassembly in a deployed configuration.

FIG. 10 illustrates a collar and lower end of a threaded shaft of a primary tightening subassembly according to the present disclosure.

FIG. 11 depicts a perspective view of a tightening knob and upper end of a threaded shaft of a primary tightening subassembly according to the present disclosure.

FIG. 12 depicts a lower perspective view of the upper parts of a primary tightening subassembly on the patient side of a device according to the present disclosure.

FIG. 13 depicts an upper perspective view of the upper parts of a primary tightening subassembly on the patient side of a device according to the present disclosure.

FIG. 14 depicts a close-up cross-sectional view of a user feedback mechanism of according to the present disclosure.

FIG. 15 illustrates a top view of the present invention in a deployed state.

FIG. 16 depicts an upper perspective view of the lower parts of a primary tightening subassembly on the patient side of a device according to the present disclosure.

FIG. 17 depicts a lower perspective view of the lower parts of a primary tightening subassembly on the patient side of a device according to the present disclosure.

FIG. 18 depicts a perspective view of a spool subassembly of the present invention with the top cover removed.

FIG. 19 depicts a perspective view of a spool subassembly of the present invention with the top cover and spool removed.

FIG. 20 is a perspective exploded view of one side of the internal parts of a spool subassembly according to the present disclosure.

FIG. 21 depicts a perspective view of a spool subassembly of the present invention with the bottom cover removed.

FIG. 22 depicts a lower perspective view of a spool subassembly according to the present disclosure.

FIG. 23 illustrates a perspective view of an embodiment of an over-pressure slipring between the shaft and primary tightening knob according to the present disclosure.

### DETAILED DESCRIPTION OF THE EMBODIMENTS

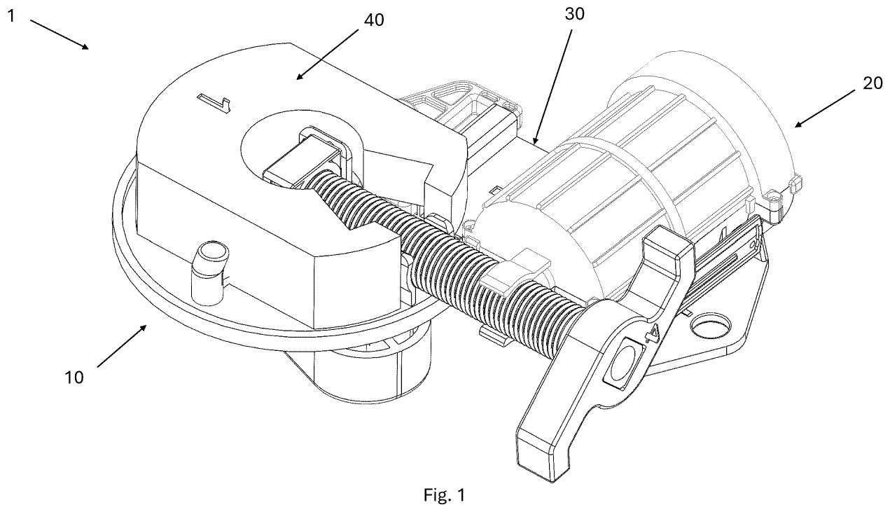

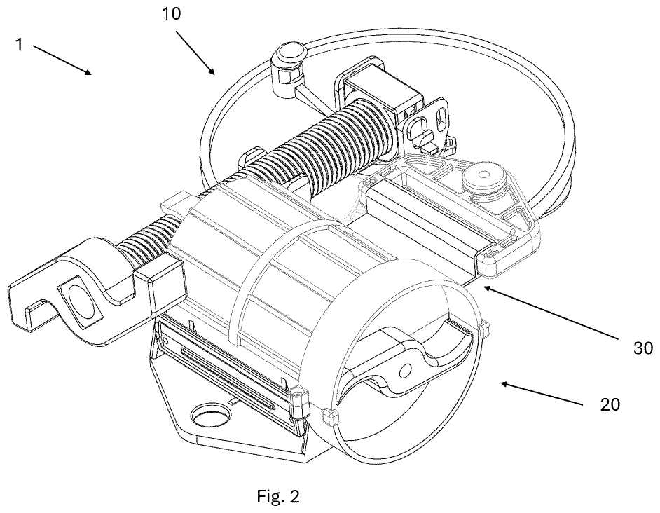

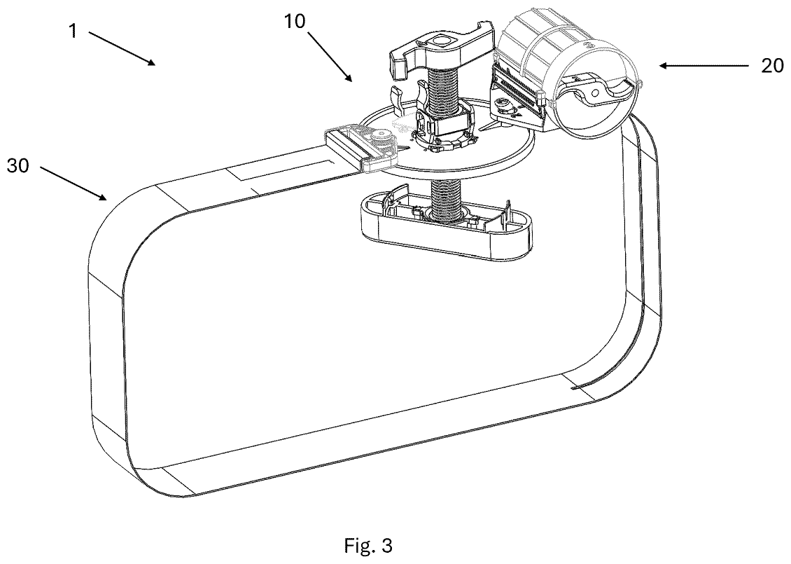

In reference to FIGS. 1-2, the user-friendly junctional tourniquet device 1 of the present invention has a stowed configuration to facilitate storage and carrying the device. FIG. 1 shows that the full device 1 comprises a primary tightening subassembly 10, strap spool subassembly 20, tourniquet strap 30, and manual pressure pad 40, and FIG. 2 shows the same full device with pressure pad 40 removed. The majority of the figures submitted herewith show pressure pad 40 removed for better visibility of other parts of device 1. FIG. 3 illustrates a preferred embodiment in a deployed state, wherein tourniquet strap 30 is configured to wrap around a body (not shown) with primary tightening subassembly 10 partially tightened to deliver some pressure. It may not be readily clear which end of tourniquet strap 30 is fixed and which end is configured to wrap around the body to form the loop. This is because either end of tourniquet strap 30 could be fixed or free according to the present invention. While preferred embodiments may have spool subassembly 20 located proximally to the free end of tourniquet strap 30, this is not limiting and an alternative embodiment could have spool subassembly 20 fixed to primary tightening subassembly 10 and the opposite end of tourniquet strap 30 may then remain free.

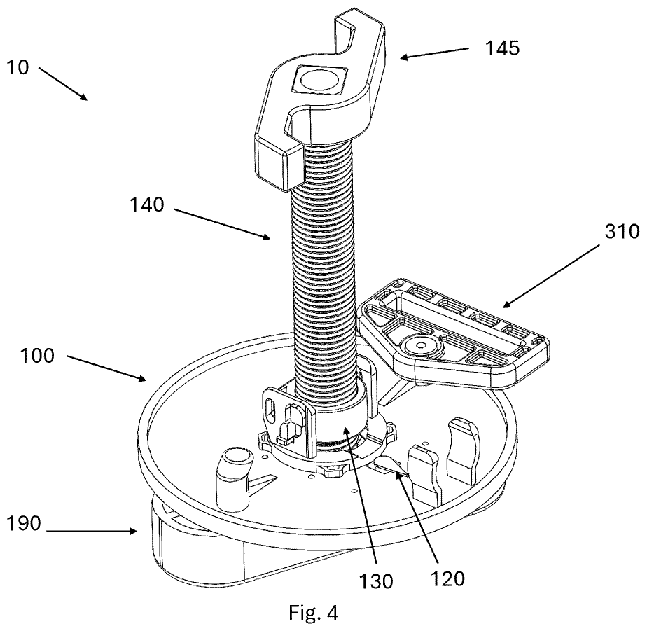

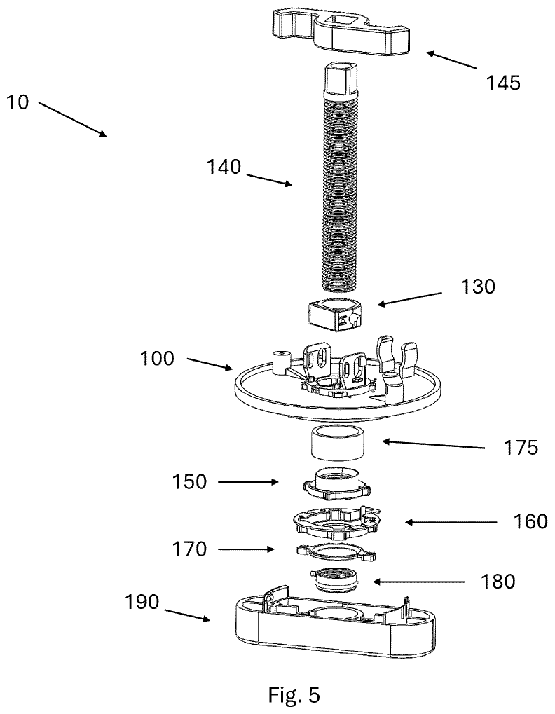

FIG. 4 shows a top perspective view of a primary tightening subassembly 10 of a preferred embodiment of the present invention and FIG. 5 shows an exploded view the primary tightening subassembly 10 of FIG. 5. Base plate 100 is the part of primary tightening subassembly 10 upon which the other parts of the subassembly are mounted. On the user side of base plate 100, there is collar 130, threaded shaft 140, and primary tightening knob 145. On the patient side of base plate 100, there is spring element 175, load transfer element 150, load transfer cap 160, retainment ring 170, ball joint 180, and pressure pad 190 for applying direct pressure to a patient. Note that pressure pad 190 is different than manual pressure pad 40, which is on the user side of base plate 100, but not shown in FIGS. 4 and 5.

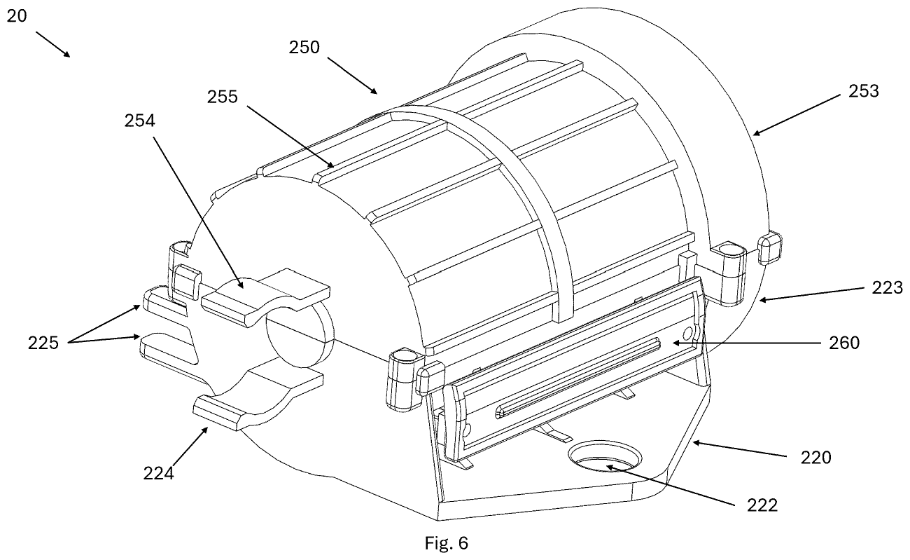

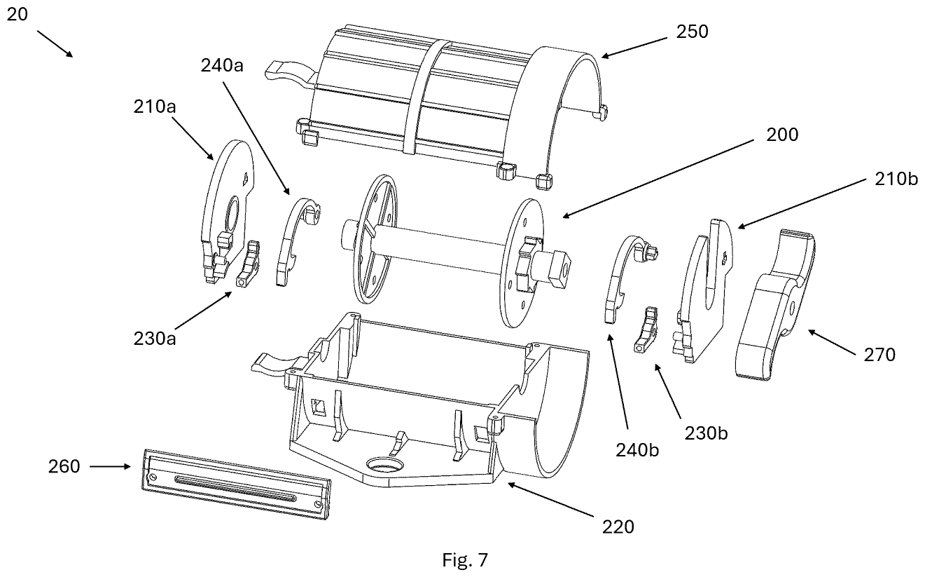

FIG. 6 shows a perspective view of spool subassembly 20 of a preferred embodiment of the present invention and FIG. 7 shows an exploded view of the spool subassembly 20 of FIG. 6. As shown in FIG. 7, spool subassembly 20 is centered around spool 200, which is held in place by carriages 210a and 210b, which are mounted between spool housing base 220 and spool housing top 250. Ratchet pawls 240a and 240b prevent tourniquet strap 30 from unwinding in a manner that loosens it. Pawl release levers 230a and 230b are included, along with pawl release bar 260, to allow a user to purposefully withdraw a length of strap 30 from spool subassembly 20 when desired. A slack removal knob 270 is also included and preferably attached to spool 200.

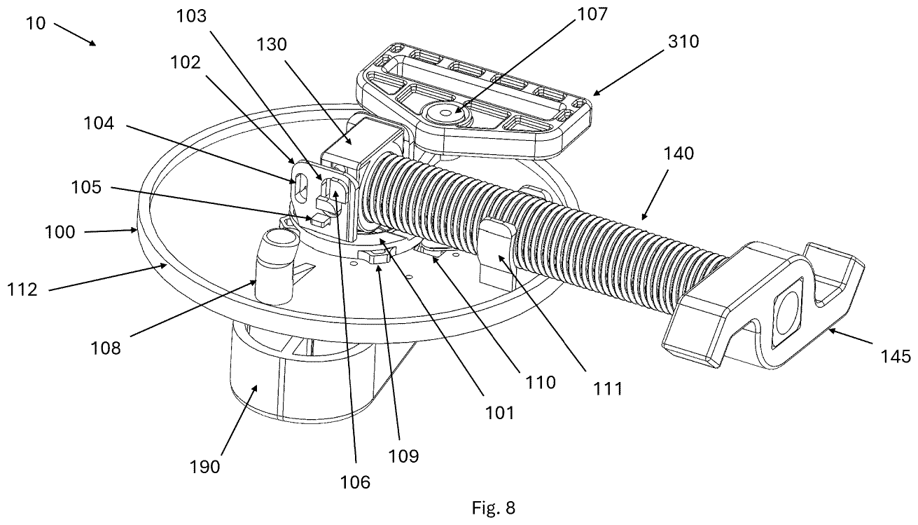

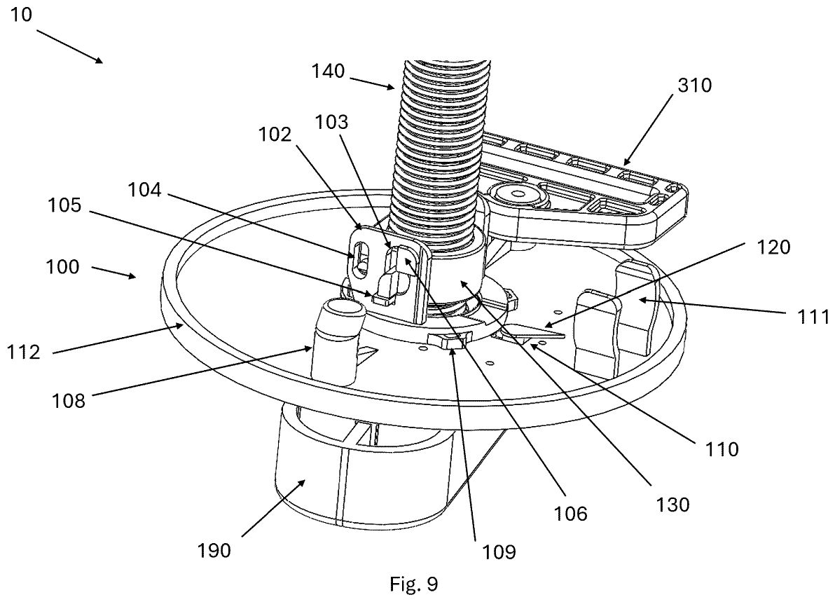

FIG. 8 shows a user side view of primary tightening subassembly 10 with manual pressure pad 40 removed, in a stowed configuration and FIG. 9 shows the same in a deployed configuration. Base plate 100 is generally a flat shape that may have a variety of stiffening features along its surfaces, such as rim 112. It has an internal passage 101, through which threaded shaft 140 interacts from the user side to components on the patient side, such as ball joint 180 and pressure pad 190. In the preferred embodiment, base plate 100 is circular and internal passage 101 is circular and centered within base plate 100, though these are not limiting to the invention.

Two vertical extensions 102 rise from base plate 100 near internal passage 101 and are situated on opposite sides of threaded shaft 140. Each vertical extension 102 has a primary slot 103 therethrough, in which collar 130 can rotate and translate a limited amount. Vertical extensions 102 may have features that serve as guides on movement of collar 130 therein. One example from a preferred embodiment is a secondary slot 104 that can interact with a feature of collar 130 to stop its rotation once threaded shaft 140 is vertical and through which vertical translation of collar 130 is limited by the length of secondary slot 104. The length of vertical travel available in secondary slot 104 is preferably the same as that available in primary slot 103. Another example from a preferred embodiment is limit-stop 105 that abuts a feature of collar 130 once it has rotated such that threaded shaft 140 is vertical. In this case, threaded shaft 140 cannot be rotated upward from base plate 100 any more than 90 degrees. Yet another example is translation guide 106, which serves to prevent rotation of collar 130 and threaded shaft 140 in the direction back to horizontal once they start to translate vertically relative to base plate 100. The invention does not require this many of such features, but the preferred embodiment has multiple, redundant features to ensure that collar 130 and threaded shaft 140 can only move in the intended manner.

On opposite sides of base plate 100 are strap starting anchor 107 and strap ending anchor 108. In a preferred embodiment these anchors are in line with internal passage 101, and when viewed from above, their alignment is perpendicular to the stowed position of threaded shaft 140. However, this arrangement and alignment is not required for the invention to operate. Strap starting anchor 107 preferably interfaces with strap starting buckle 310, which is pivotably attached thereto in a fixed manner. That is, a user is preferably not intended to connect or disconnect base plate 100 and strap starting buckle 310. In this configuration, tourniquet strap 30 is permanently attached to strap starting buckle 310 at one end and permanently attached to spool 200 at its other end, and the majority of its length is wound around spool 200 in the stowed configuration. After the user wraps tourniquet strap 30 around the patient, a feature on spool housing base 220 or spool housing top 250 attaches to strap ending anchor 108 to form a complete loop around a patient. In a preferred embodiment, strap ending anchor 108 is somewhat hook-shaped with the opening of the hook oriented toward internal passage 101, though this is not a limitation of the invention. For example, an alternative embodiment may include any attachment means and still be within the metes and bounds of the claimed invention. Examples include but are not limited to buckle and tongue/latch plate features common in car seat belts, side release buckles, center release buckles, cam buckles, magnetic buckles, and snap hooks.

The arrangement described above for which end of tourniquet strap 30 is fixed to base plate 100 can be reversed in an alternate embodiment, as one skilled in the art will readily understand. That is, strap starting anchor 107 could alternately be pivotably attached to a feature on spool housing base 220 or spool housing top 250 in a fixed manner, such that the user is preferably not intended to connect or disconnect base plate 100 from spool subassembly 20. In this embodiment, strap starting buckle 310 then becomes strap ending buckle 310, as the end of tourniquet strap 30 attached thereto is the end that is wrapped around the patient and it is attached to base plate 100 via strap ending anchor 108 to form a complete loop around the patient.

In line with the stowed position of threaded shaft 140 are retainment clips 111, with one being on each side of the shaft. The purpose of retainment clips 111 is to hold threaded shaft 140 in the horizontal position, against base plate 100, while it is being stowed. Retainment clips 111 are preferably of the same material as base plate 100, formed as part of base plate 100, and extend vertically therefrom, though none of this is required for the invention to be operable.

Base plate 100 also preferably has an opening 110 to allow passage of user feedback features from the patient side to the user side of the plate. In the embodiment shown in FIG. 9, user feedback device 120 has a pointer visible from above, though any such feedback mechanism is within the spirit of the invention. Other examples include but are not limited to needle gauges and slider gauges.

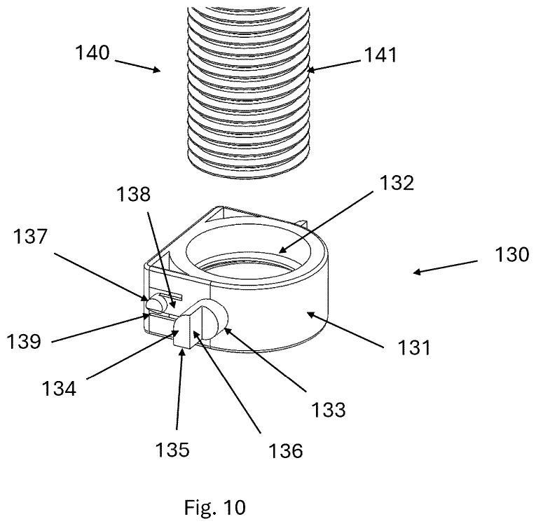

Collar 130 is shown in FIG. 10, along with the lower end of threaded shaft 140. Collar 130 is preferably and generally of a cylindrical ring shape. Collar body 131 has at least a partial thread 132 on its inner face which interfaces with threads 141 of threaded shaft 140. Thread 132 is used to keep threaded shaft 140 engaged as part of primary tightening mechanism 10 and is preferably stowed with more than one turn of engagement. Collar 130 interfaces with base plate 100 via its lateral posts 133 that extend outward from body 131 and on opposite sides. Lateral posts 133 are constrained to move within primary slots 103 of base plate 100. At the lower end of primary slots 103, lateral posts 133 are free to rotate. Beginning from the stowed horizontal position of threaded shaft 140, collar 130 rotates freely up to the vertical position (i.e., perpendicular to base plate 100).

Once threaded shaft 140 is vertical, collar 130 is prevented from rotating any more. In the preferred embodiment, there are two (redundant) features that prevent further rotation. Lateral posts 133 have post extensions 134 that extend laterally outward and do not keep the circular shape of lateral posts 133. At the perpendicular position, post extensions 134 have a flat surface that is horizontal (i.e., parallel to base plate 100) that abuts limit-stops 105. Additionally, when threaded shaft 140 is in the vertical position, secondary slots 104 of base plate 100 engage with pegs 137 of collar 130. Pegs 137 are preferably cantilevered at the end of flexible beams 138 such that they flex inward toward threaded shaft 140 when they come into contact with vertical extensions 102 and remain inwardly flexed until pegs 137 are freed by secondary slots 104, wherein they snap into place. Pegs 137 preferably also have angled surfaces 139 on the side that first contacts vertical extensions 102 during their rotation from horizontal to vertical (in reference to threaded shaft 140 positions) to facilitate and help initiate the bending of flexible beams 138. At this point (vertical alignment of threaded shaft 140), collar 130 cannot rotate any more, but it is now free to translate vertically upward relative to base plate 100, as pegs 137 slide within secondary slots 104 and vertical guide surface 136 is therein restrained by translation guides 106 such that lateral posts 133 slide within primary slots 103. While limit-stops 105 and translation guides 106 are used with primary slots 103, and cantilevered pegs 137 are used with secondary slots 104 in the preferred embodiment, these examples are not meant to be limiting. One skilled in the art will readily know multiple alternative ways to impose the same rotation and translation restrictions between two parts of a device, and the specific methods used are not a distinguishing feature of the invention.

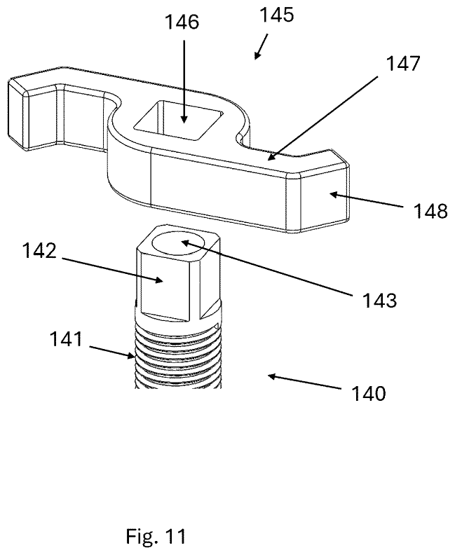

Tightening knob 145 is shown in FIG. 11, along with the upper end of shaft 140. Tightening knob 145 mounts to the upper end of threaded shaft 140. In a preferred embodiment, the mounting is accomplished via flat inner surfaces 146 of tightening knob 145 mating with flat outer surfaces 142 of threaded shaft 140, though any such mating of two parts would be within the spirit of the invention. For example, an alternative embodiment could have threads 141 of threaded shaft 140 extend to the upper end thereof and these external threads could mate with similar internal threads in tightening knob 145. While any shape of knob could be used with the invention, the preferred embodiment has a biased knob that has two arms 147 extending tangential from the center circle, rather than the common radial extension of prongs on knobs. Further, the ends of arms 147 have paddles 148 that bend back toward the centerline of the part. The preferred embodiment has tightening knob 145 biased as such to encourage a user to turn the knob in the correct (i.e., tightening) direction during use of the device.

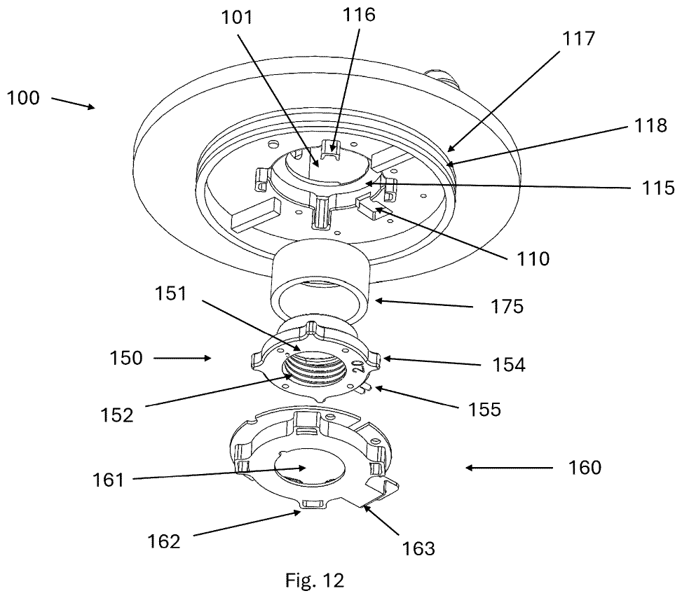

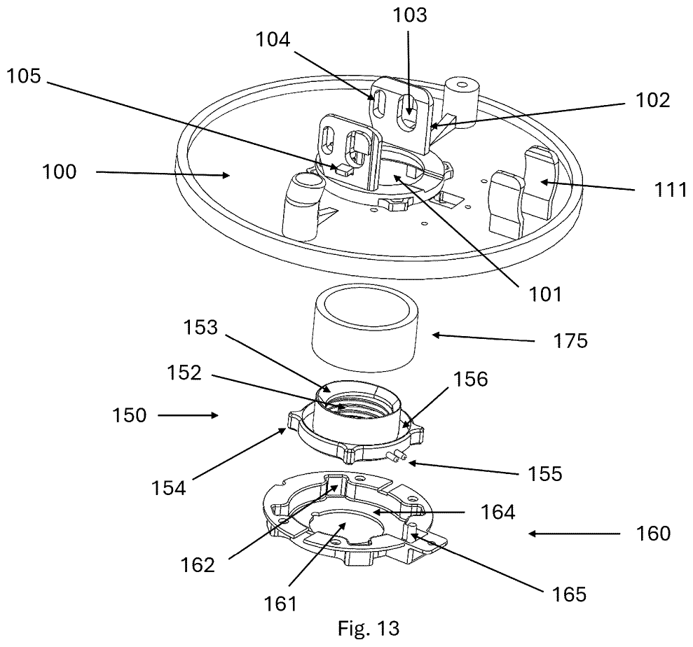

FIGS. 12 and 13 show bottom and top perspective views, respectively, of the patient side of base plate 100 with other interacting parts of the invention. Spring element 175 is restrained on the top by cavity 115 of base plate 100 and on the bottom by cavity 156 of load transfer element 150. While preferably a coil spring or foam tube, spring element 175 could be any single or series of elastic energy storage elements for the purposes of the present invention. Load transfer element 150 is likewise restrained on the top by travel channel 116 of base plate 100 and on the bottom by cavity 164 of load transfer cap 160, which itself is fastened or otherwise fixed to base plate 100. Within these constraints, load transfer element 150 is free to translate vertically when load is applied, and its motion relative to base plate 100 is proportional to the stiffness of spring element 175. Load transfer element 150 is prevented from rotating about the vertical axis of primary tightening subassembly 10 by guide tabs 154, which interface with travel channels 116. Load transfer cap 160 may also have travel channels 162 for interfacing with guide tabs 154 or the downward extension of travel channels 116 from base plate 100, as is the case for the preferred embodiment.

Load transfer element 150 has internal threads 152 that interface with threads 141 of threaded shaft 140. Unlike collar 130 that preferably has only one thread revolution 132, internal threads 152 preferably span the inside diameter of hole 151. To facilitate guidance of threaded shaft 140 into load transfer element 150 during primary tightening, load transfer element 150 preferably has a conical top 153 that acts somewhat like a funnel. Note that hole 161 of load transfer cap 160 preferably has no internal threads and allows threaded shaft 140 to pass freely therethrough.

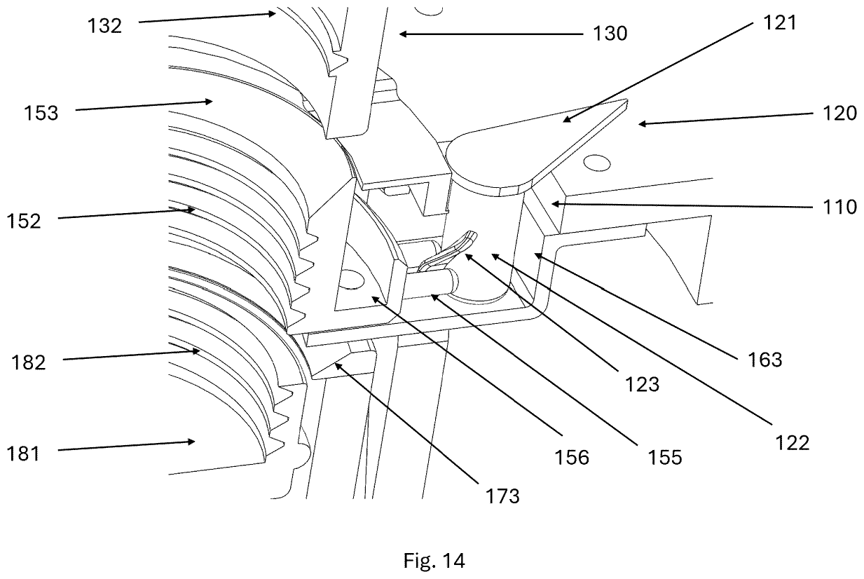

In a preferred embodiment, load transfer cap 160 has post 165 that extends vertically upward and is aligned such that it is located within opening 110 of base plate 100. Its purpose is to provide a fixed reference point for user feedback device 120. As shown in FIG. 14 for a preferred embodiment, user feedback device 120 resembles an indicator needle. The part that the user sees from above (i.e., user side of base plate 100) is pointer 121. Extending below pointer 121, through opening 110 of base plate 100 is cylindrical tube base 122, which slides over post 165 and rotates thereabout. Cylindrical tube base 122 also has helical spine 123 extending radially outward therefrom. Helical spine 123 fits between pressure posts 155 of load transfer element 150. During operation of primary tightening subassembly 10, load transfer element 150 translates vertically upward as pressure applied to the patient increases. As load transfer element 150 moves upward, pressure posts 155 traverse along helical spine 123 like a cam-follower mechanism and cause pointer 121 to rotate. From above and from the user's perspective, base plate 100 may have pressure reference 125, over which pointer 121 travels.

While a preferred embodiment as described has collar 130 and load transfer element 150 as two distinct parts that both engage with threaded shaft 140 and translate with it relative to base plate 100, an alternative embodiment has these two parts combined into a single part to simplify the mechanism and reduce part count.

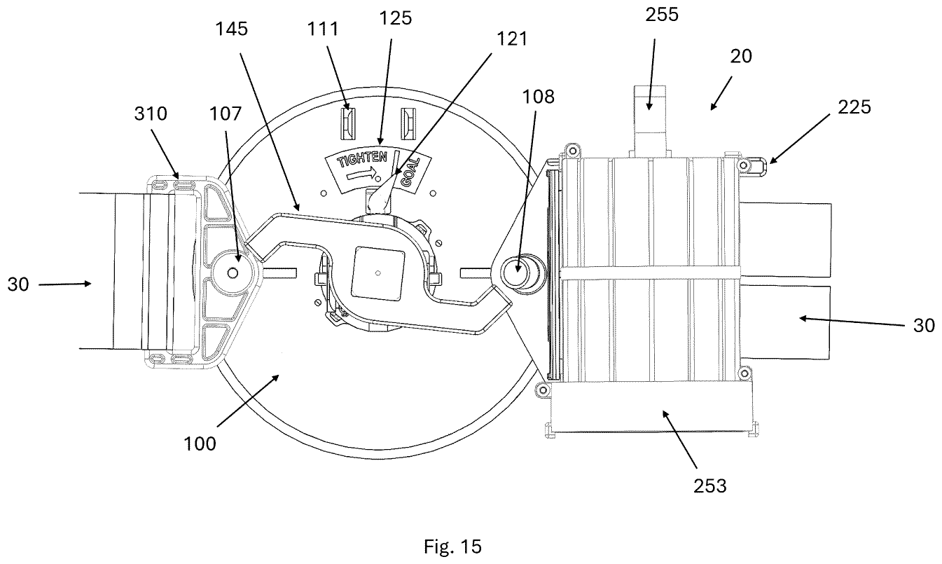

In a preferred embodiment and as shown in FIG. 15, pressure reference 125 is a decal that shows the user information relevant to tightening the device, such as gradual tightening progress and a pressure goal line, over which pointer 121 should pass before the user stops tightening. While the preferred embodiment may use a decal to display this information, any method could be used, such as stamping in onto base plate 100 or molding it directly into the part. Similarly for the activation of user feedback device 120 via movement of load transfer element 150, the preferred embodiment described a cam-follower mechanism, but this is not meant to be limiting. Anyone skilled in the art will readily understand that any variety of mechanisms could accomplish the same result, including but not limited to gear-rack, linkage, and lever mechanisms.

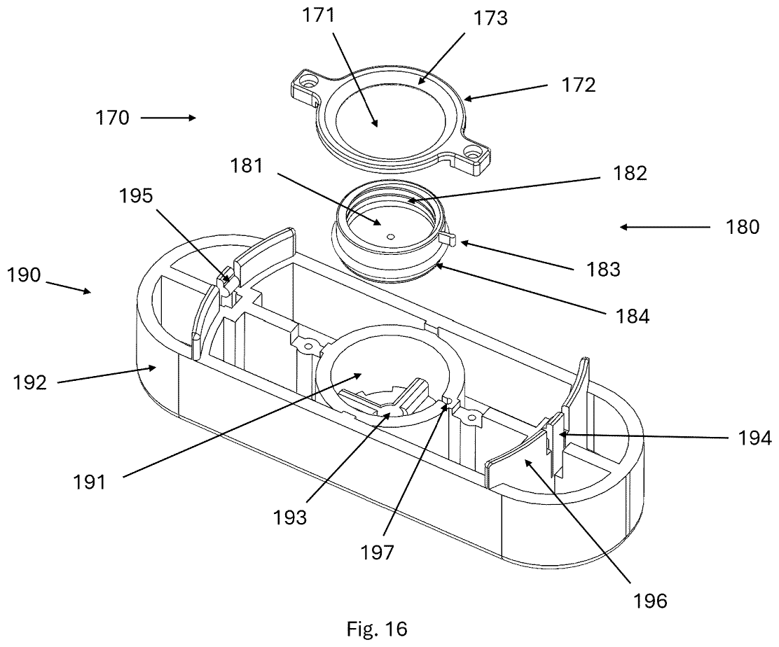

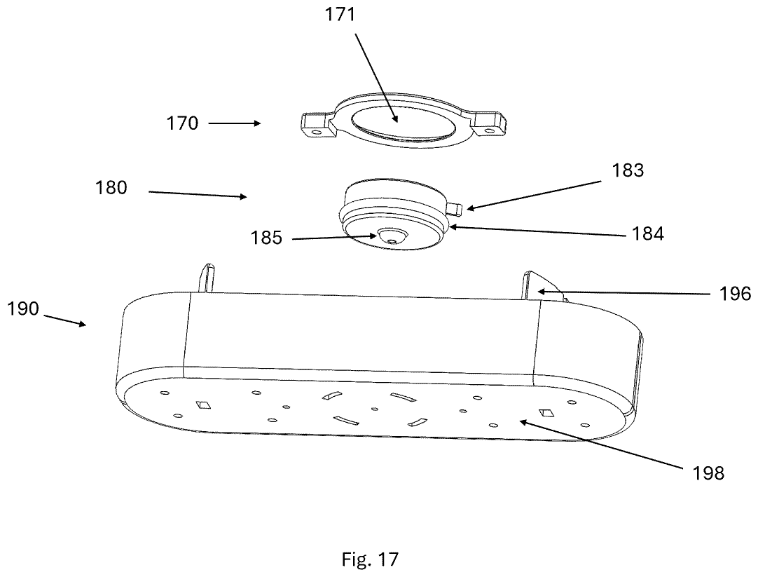

Moving farther down the chain of parts of primary tightening subassembly 10, FIGS. 16 and 17 show top and bottom perspective views, respectively, of retainment ring 170, ball joint 180, and pressure pad 190. Ball joint 180 fits within joint cavity 191 of pressure pad 190 and is held therein by retainment ring 170. Retainment ring 170 has hole 171 similar to hole 161 of load transfer cap 160 which allows free passage of threaded shaft 140 therethrough. Similar to conical top 153 of load transfer element, retainment ring 170 also has conical top 173 for the same reason of helping to guide threaded shaft 140 through it and into threads 182 of ball joint 180. Ball joint 180 is generally cup-shaped with internal threads 182 for interfacing with threads 141 of threaded shaft 140 in cup cavity 181. There is preferably more than one full revolution of threads for threads 182 before threaded shaft 140 bottoms out in cup cavity 181 as it screws in.

To facilitate engagement with threaded shaft 140, the preferred embodiment of ball joint 180 has breakaway tab 183 that fits in breakaway cavity 197 of pressure pad 190. Its purpose is to prevent rotation of ball joint 180 until threaded shaft 140 is fully screwed in, at which point continued turning of threaded shaft 140 will cause breakaway tab 183 to break away from ball joint 180. Note that breakaway tab 183 is held within the device by its own shape and fit within breakaway cavity 197, as well as by retainment ring 170. The continued turning of threaded shaft 140 will begin to push pressure pad 190 away from base plate 100. Ball joint 180 turns relative to pressure pad 190 at this point and may be facilitated by a feature such as ring 184 which reduces surface area and friction during this relative movement. As downward force is being applied on primary tightening subassembly 10, hemisphere 185 on the bottom of ball joint 180 reduces surface area and friction with pressure pad 190 as it spins on platform 193. Ring 184 and hemisphere 185 also allow pressure pad 190 some freedom to pivot out of plane relative to base plate 100, as may be required to better conform with the body of the patient.

Pressure pad 190 generally has smooth patient contact surface 198 with rounded edges for applied direct pressure to the patient. In addition to the walls of joint cavity 191, side walls 192 provide rigidity to pressure pad 190 to ensure efficient delivery of pressure. In the preferred embodiment, the radial extents of patient contact surface 198, and pressure pad 190 itself, match the radius of base plate 100. In the stowed configuration and before turning of threaded shaft 140 forces pressure pad 190 away from base plate 100, pressure pad 190 is held onto base plate 100 by detents 195 at the top of cantilevers 194. Detents 195 fit within detent channel 118 of bottom ring 117 on base plate 100. In the preferred embodiment, bottom ring 117 is a full circle and detent channel 118 is an annular cavity therein. Pressure pad 190 can be manually rotated relative to base plate 100 when detents 195 are engaged to allow the user to adjust the orientation of pressure pad 190 on the patient. Adjustment guides 196 may also be included to facilitate this rotation adjustment, as well as offer protection to cantilevers 194 and detents 195. Cantilevers 194 are designed to provide some resistance to separation from base plate 100 but will bend out of the way once a certain force is applied via turning threaded shaft 140.

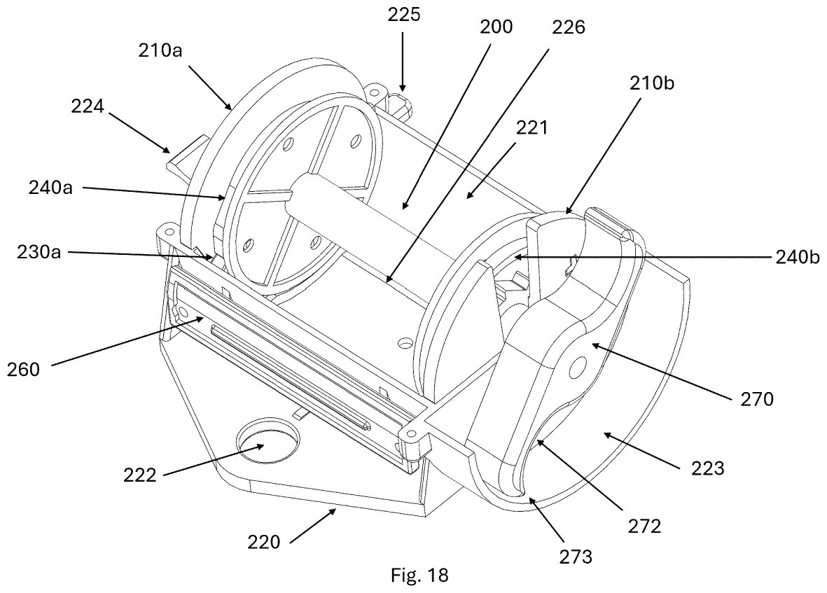

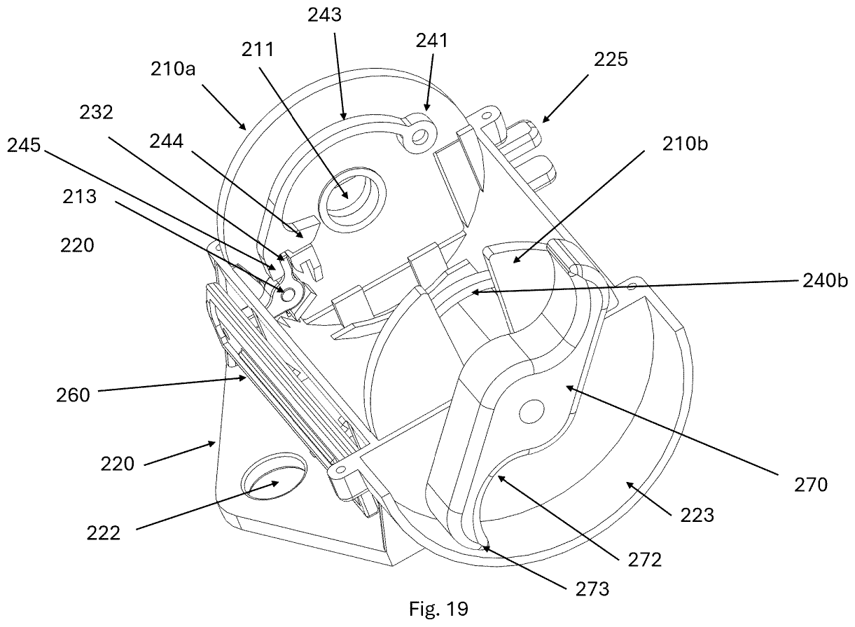

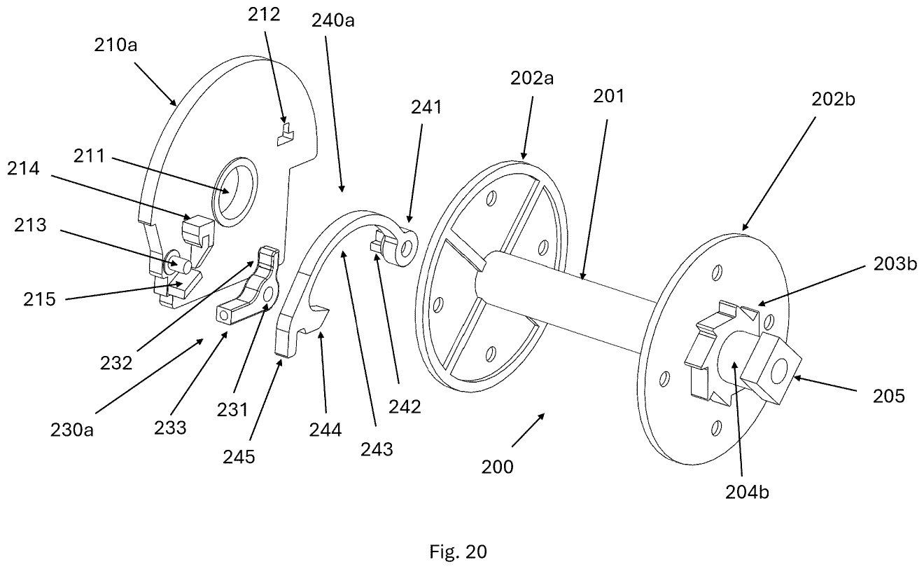

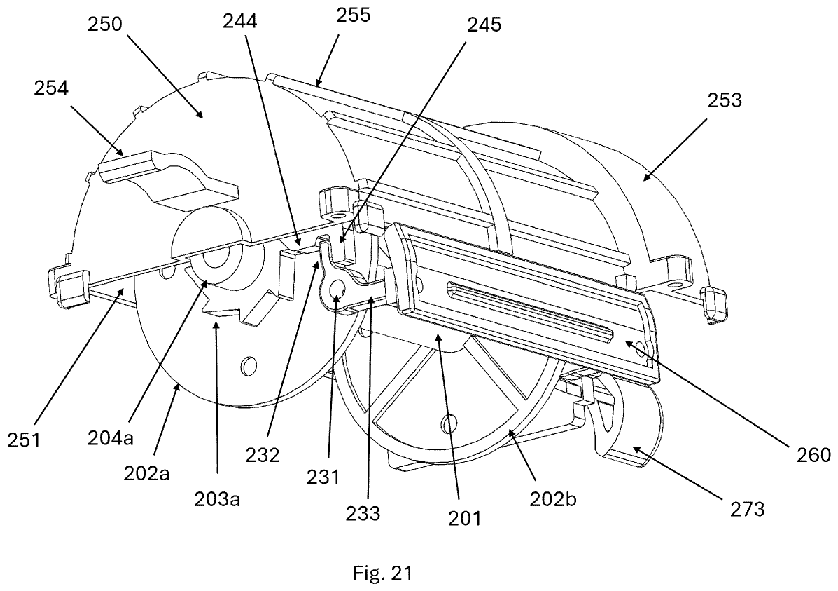

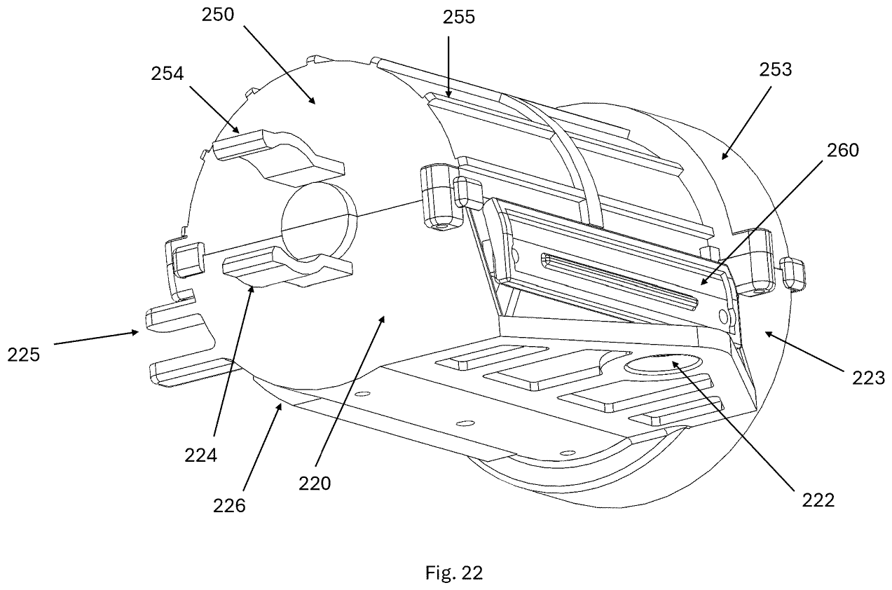

FIG. 18 shows a view of spool subassembly 20 of the present invention, with spool housing top 250 removed. Key parts of the subassembly identified here are spool 200, spool carriages 210, spool housing base 220, pawl release levers 230, ratchet pawls 240, release bar 260, and slack removal knob 270. FIG. 19 shows a slightly different angle with spool 200 also removed. FIG. 20 shows an exploded view of spool 200 and one side of the carriage-pawl-release parts. Note that the opposite side of spool 200 has similar parts that are nearly mirror images of these. FIG. 21 shows a lower perspective view of spool subassembly 20 with spool housing base 220 removed. FIG. 22 shows a lower perspective view of spool subassembly 20.

While one end of tourniquet strap 30 terminates at strap starting buckle 310, the other terminates around spool center 201 of spool 200. Tourniquet strap 30 wraps around spool center 201 several times and is contained inside spool housing base 220 and spool housing top 250, exiting from the covers via strap slot 226 of spool housing base 220. Opposite strap slot 226 is base plate engagement loop 222, which is placed over ending anchor 108 to complete a loop around a patient.

Tourniquet strap 30 is wound between discs 202. Outward from discs 202 on spool 200 are ratchet gears 203 and bearing extensions 204, and on one end mating surfaces 205 for connection with slack removal knob 270. The default, resting configuration of spool subassembly 20 has tourniquet strap 30 locked from unwinding (i.e., loosening) via position of ratchet gears 203 and ratchet pawls 240. In this configuration, slack removal knob 270 can be turned clockwise to wind more strap into the housing. However, it cannot be turned counterclockwise to release more strap from the housing, nor can pulling on tourniquet strap 30 result in unwinding additional length from spool 200. This is because foot 244 of ratchet pawl 240 interferes with ratchet gear 203.

When spool 200 winds (or unwinds), bearing extensions 204 rotate within bearing surfaces 211 of spool carriages 210, with spool carriages 210 themselves being held fixed within spool housing base 220 and spool housing top 250. Spool carriages 210 provide an anchor point for ratchet pawls 240 via pawl interface 212, which mates with pawl anchor features 242. They also provide a limit-stop 214 for pawl foot 244 to prevent unwanted rotation of spool 200. Pawl anchor features 242 extend from pawl base 241 laterally. Extending from pawl base 241 circumferentially is curved cantilever 243, which terminates at its other end with foot 244 and heel 245. To unwind a length of tourniquet strap 30 from spool 200, a user must depress pawl release bar 260, which is attached to pawl release levers 230 via bar arm 233. Depressing pawl release bar 260 causes pawl release lever 230 to pivot about its mounting hole 231, which is assembled over pawl post 213 on spool carriage 210. Such rotation of pawl release lever 230 causes pawl arm 232 to push generally radially outward on heel 245 such that foot 244 moves out of contact with ratchet gear 203. Then spool 200 can freely unwind. Release limiters 215 may be included to provide guidance on depression of pawl release bar 260.

Spool housing base 220 and spool housing top 250 have primary cavities 221 and 251, respectively, in which the internal parts described above and tourniquet strap 30 reside. On their knob ends, they have shrouds 223 and 253, respectively, which offer protection to slack removal knob 270. On their opposite ends, they preferably have shaft retainment clips 224 and 254, respectively. Similar to retainment clips 111 on base plate 100, shaft retainment clips 224 and 254 hold spool subassembly 20 to threaded shaft 140 in the stowed configuration. A preferred embodiment also has base plate retainment clips 225 extending from spool housing base 220 for engagement with base plate 100 in the stowed configuration. These clips are intended to keep the parts of the device held together when stowed and packaged to make deployment and use easier for the user. The spool housing parts may also have grip enhancements 255 to facilitate handling of spool subassembly 20 with gloved hands and/or when fluids are present.

Slack removal knob 270 is preferably shaped similar to primary tightening knob 145. It is preferably two-pronged with biased arms 272 that are tangential to its body, and at the end of biased arms 272 are paddles 273 that are angled back toward the central axis of the part. Recall that this is to encourage rotation of the knob in the correct direction. An alternative embodiment includes a spring (e.g., power spring) in place of slack removal knob 270, such that energy is stored in the spring as tourniquet strap 30 is pulled out by the user and that stored energy is released when the user releases tourniquet strap 30 and the slack is automatically removed by recoiling back into spool housing base 220 and spool housing top 250. Yet another alternative embodiment may include both a recoil spring and a slack removal knob 270.

Below is a detailed description of how a user completes the steps to apply device 1 to a patient in need. A preferred embodiment is used as an example but is not intended to be limiting in the invention. One skilled in the art will readily understand how the steps are similarly completed in alternate embodiments.

The first step of the application procedure is to apply direct pressure to the application site, either inguinal or axillary. A preferred embodiment comes out of its package stowed as a single unit. It can be used directly to apply pressure to the patient. The patient side of the base plate 100 has pressure pad 190 that is rotationally adjustable for placement either along the inguinal junction or just below the clavicle. The user applies direct pressure (e.g., with the hand or knee if the patient is lying supine) via manual pressure pad 40 on the user side of base plate 100. It may be marked with the number “1” to start the user thinking about sequential steps for this user-friendly device 1. This manual pressure is maintained to the extent possible while the user completes steps 2 and 3. Note that this early and largely uninterrupted pressure delivery is an improvement over the current market offerings which generally require the user to interrupt pressure application while applying the tourniquet. In the hands of the untrained or minimally trained user, the time it takes to apply a currently available junctional tourniquet (with no pressure being applied) would translate to significant blood loss. In addition, a hemostatic dressing or other wound packing material may be carried with user-friendly junctional tourniquet device 1. Note that the bottom (patient) side of device 1 is relatively flat and pressure pad 190 can be rotated to create a pocket between pressure pad 190 and primary tightening knob 145 in the stowed configuration, where a dressing can be stored.

The second step of the procedure is to fasten tourniquet strap 30 around the patient with base plate 100 remaining where it was placed in step 1. To complete this step, spool subassembly 20 may be unclipped from threaded shaft 140 (via 224 and 254) and from base plate 100 (via 225) by applying a nominal force. Then spool subassembly 20 can be pivoted about strap starting buckle 310 into position for looping tourniquet strap 30 around the patient. Note that this can be done while still maintaining direct manual pressure via manual pressure pad 40 so as not to interrupt pressure delivery and to maintain hemorrhage control during device preparation/application. Then the user depressed pawl release base 260 and pulls on spool subassembly 20 like a seat belt to deploy a length of tourniquet strap 30 that wraps around the patient. Differing from a seat belt though, tourniquet strap 30 is not spring loaded in the preferred embodiment, as this would make passing it behind the patient more difficult. Instead, there is nominal resistance to withdrawing tourniquet strap 30 from spool subassembly 20 via internal friction. An alternate embodiment may include a spring-loaded strap. Another alternate embodiment may include a more seatbelt-like mechanism where the user pulls on the buckle tongue/latch plate.

After passing tourniquet strap 30 behind the patient, the user then places base plate engagement loop 222 of spool subassembly 20 over strap ending anchor 108 on base plate 100. For usability, the mating components may both be numbered “2” and the user is essentially aligning the number “2” on each of the device components. The shape of strap ending anchor 108 and proximate its proximate position to manual pressure pad 40 will prevent the two parts from separating but remain simple and easy to connect if only one hand is available. This is also an improvement over current market offerings, which generally require two hands or are more difficult to do with a single hand. An alternate embodiment may include a more seatbelt-like tongue and buckle for this step.

Note that for axillary placement, the manual pressure may have to be temporarily reduced while passing tourniquet strap 30 behind the shoulders, but the position of pressure pad 190 and the user's hand on manual pressure pad 40 can be maintained. Also note that the fixed end (via 107 and 310) of tourniquet strap 30 may be split lengthwise to facilitate securement of the device around a patient in the axillary position-part of tourniquet strap 30 can go around the arm or side of the torso and the other part of the strap can go above and around the clavicle. This split end design has no negative effect on inguinal placements and may be an improvement over existing devices.

The third step of the procedure with the preferred embodiment is to remove slack from tourniquet strap 30 after it has been fastened around the patient. Once base plate engagement loop 222 has been connected to strap ending anchor 108 on base plate 100, the user's hand can simply slide to the side of spool subassembly 20 where slack removal knob 270 resides for removing slack. The user turns slack removal knob 270, labeled with the number “3” in this embodiment to wind any extra deployed length of tourniquet strap 30 back onto spool 200. Note that in an embodiment, an internal mechanism to the housing may have this slack winding function de-coupled initially from the primary strap spool 200 so it does not interfere with the strap deployment for wrapping around the patient. In this case, then pulling on the handle creates a force that engages or couples the primary spool to enable the rewinding action. One method of achieving this coupling/decoupling switch is to use a pair of ratchet pawls engaged/disengaged from the primary spool during the application procedure so that initially the deployed strap would stay out for wrapping around a patient (i.e., anti-tightening) and then it switches to where the strap stayed in the housing so it could not be pulled out any farther (i.e., anti-loosening). The type of mechanism used here is not intended to be limiting of the invention, however, as one skilled in the art will be familiar with alternate methods and/or mechanisms to achieve the same results, and these are considered to be within the spirit of the invention.

Recall that in an alternative embodiment slack removal knob 270 may be replaced with a spring that stores energy as primary strap spool 200 unwinds and a length of tourniquet strap 30 is pulled out by the user. Then once the loop around the patient is completed by attaching the free end of tourniquet strap 30 to base plate 100, the user releases tourniquet strap 30 and slack is automatically removed as stored energy in the spring releases. Another alternative embodiment includes a combination of both the spring and slack removal knob 270.

The fourth step of applying the preferred embodiment of the user-friendly junctional tourniquet device 1 is to replace the manually applied pressure with final device-applied pressure via turning primary tightening knob 145. Threaded shaft 140 is first disengaged from retainment clips 111 of base plate 100 by applying a nominal force and then rotated to the vertical position (perpendicular to base plate 100) where it preferably clicks into position to ensure alignment of the components of primary tightening subassembly 10 along the central axis through hole 101. Primary tightening knob 145 is now turned to advance threaded shaft 140 through base plate 100A, into engagement with load transfer element 150, through load transfer cap 160 and retainment ring 170, and into engagement with ball joint 180. Note that the screw mechanism here avoids pressure variations resulting from environmental changes (e.g., temperature, altitude) that decrease the effectiveness of devices with a pneumatic component. Primary tightening knob 145 is preferably labeled with the number “4” to indicate its place with the sequence of user steps. Primary tightening knob 145 is preferably of a biased prong design to make it more difficult to turn in the wrong (counterclockwise) direction in cases where the user is confused or under situational duress. Note that in the preferred embodiment, user feedback device 120 and pressure reference 125 are initially under threaded shaft 140 in the stowed configuration and become visible to the user when bringing threaded shaft 140 to the vertical position.

In an alternate embodiment, primary tightening knob 145 may contain user feedback device 120 instead of base plate 100 to provide real-time user feedback regarding tightening progress. In this embodiment relevant components of primary tightening subassembly 10 below base plate 100 would communicate with user feedback device 120 through internal pass-through 143 of threaded shaft 140. Regardless of the location of user feedback device 120, the user is intended to turn primary tightening knob 145 until pointer 121 reaches the “target zone” or other indicated level of pressure-on-pressure reference 125.

There are numerous ways in which user feedback device 120 can be driven, the details of which are not intended to be limiting of the invention. For example, a spring-loaded mechanism may be used with a lever mechanism to drive the needle motion on the gauge in one embodiment, while another embodiment may include different mechanisms, such as cam/follower or gears that also fall within the spirit of the invention. In the preferred embodiment, spring element 175 may initially be tuned/selected for pointer 121 entering the target zone at 200 mmHg and maxing it out at 300 mmHg, as this is generally expected to be sufficient to occlude flow at both the axillary and inguinal locations based on other junctional tourniquet studies, though different pressures could be used without changing the invention.

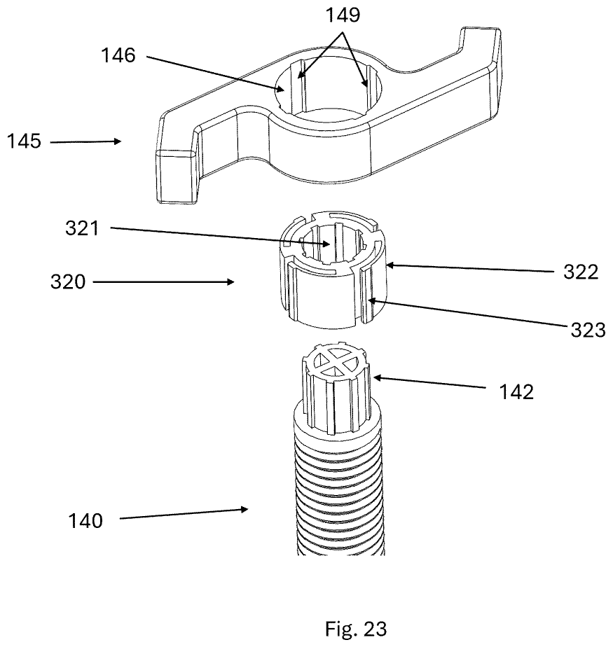

As an added safety feature, primary tightening knob 145 may be fitted with over-pressure slip-ring 320 at the interface between primary tightening knob 145 and threaded shaft 140, such that torques applied to primary tightening knob 145 that are over the pre-defined limit may cause it to rotationally slip relative to threaded shaft 140, thereby not transferring that higher torque to continued tightening/pressure application to the patient. One such example of how this could be accomplished is illustrated in FIG. 23, which shows an exploded view of the top of threaded shaft 140. Here flat inner surfaces 146 have changed from generally square shaped to generally circular shaped with slip-ring engagements 149 spaced circumferentially around. In this example, there are four such engagements but this is not meant to be limiting. Similarly, the top end of threaded shaft 140 (flat outer surfaces 142) could change its shape to match inside mating surface 321 of slip-ring 320. Note that differently sized (i.e., smaller) flat outer surfaces 142 would also work, but a different shape was illustrated here for variety, as it is not critical to the invention. The preferred embodiment of slipring 320 is generally circular with circumferential cantilever segments 322 that have feet 323 at their free end. Feet 323 reside within slip-ring engagements 149 under normal/allowable torques, but when excessive torque is applied to primary tightening knob 145, circumferential cantilever segments 322 bend inward and primary tightening knob 145 slips its position relative to threaded shaft 140 until feet 323 catch in the next circumferential engagement 149.

The fifth and final step of the procedure with the preferred embodiment is then to simply monitor pointer 121 relative to pressure reference 125 periodically. If pointer 121 is observed to have fallen below the target zone indicated on pressure reference 125, the user is to re-tighten device 1 by turning primary tightening knob 145 again until pointer 121 re-enters the target zone, indicating that an acceptable level of pressure is being applied to the patient. Alternatively, if the site of bleeding is fully visible and the user is confident that hemorrhage has been controlled, the needle location at the point of hemorrhage control may be noted. Thereafter, the user may refer to the noted gauge needle position and re-tighten as needed to maintain occlusion. The gauge face (i.e., pressure reference 125) may include hash marks around its perimeter for use in such instances.

Having now fully set forth the preferred embodiments and certain modifications of the concept underlying the present invention, various other embodiments as well as certain variations and modifications thereto may obviously occur to those skilled in the art upon becoming familiar with the underlying concept. It is to be understood, therefore, that the invention may be practiced otherwise than as specifically set forth herein.

What is claimed is:

1. A tourniquet device for restricting flow of blood in a junctional location of a patient, said tourniquet device comprising:
a strap for wrapping around the body of a patient proximate to said junctional location, said strap having a first end and a second end, said first end being fixed to a buckle end and said second end being fixed to a spool, said spool configured for winding and unwinding said strap by rotating about its longitudinal axis;
a base plate having a patient side and a user side for coupling said first end to said second end to form a loop around the body of a patient, said base plate configured for attachment of said buckle end and said spool;
at least one ratchet gear and ratchet pawl configured in a resting state to be engaged to prevent said strap from unwinding from said spool;
at least one pawl release lever configured to disengage said at least one ratchet gear and ratchet pawl to allow said strap to unwind from said spool;
a collar pivotably and translatably attached to said base plate to mechanically engage with a threaded shaft, said threaded shaft configured to mechanically engage with a pressure pad to apply pressure to a patient as said threaded shaft is rotated about its longitudinal axis, said pressure pad being rotationally and releasably mounted to said patient side of said base plate;
a load transfer element configured to engage and translate with said threaded shaft relative to said base plate;
a spring element acting between said base plate and said load transfer element and configured to control translation of said threaded shaft relative to said base plate in response to pressure being applied to a patient; and
a user feedback mechanism movably coupled to said load transfer mechanism and configured to provide a real-time indication to a user of pressure applied to a patient.

2. The tourniquet device of claim 1 wherein said buckle end is pivotably fixed to said base plate and said spool is configured to releasably attach to said base plate.

3. The tourniquet device of claim 1 wherein said spool is pivotably fixed to said base plate and said buckle end is configured to releasably attach to said base plate.

4. The tourniquet device of claim 1 further comprising a ball joint interface between said threaded shaft and said pressure pad.

5. The tourniquet device of claim 1 further comprising a manual pressure pad on said user side of said base plate.

6. The tourniquet device of claim 1 further comprising a tightening knob configured to facilitate rotating of said threaded shaft and a torque slip-ring interface between said threaded shaft and said tightening knob.

7. The tourniquet device of claim 1 further comprising at least one retaining clip configured to releasably hold said threaded shaft in a stowed position.

8. The tourniquet device of claim 1 further comprising at least one retaining clip configured to releasably hold said base plate.

9. The tourniquet device of claim 1 wherein said collar restrictedly pivots approximately 90 degrees from a stowed configuration to an active configuration, said active configuration enabling rotation of said threaded shaft about its longitudinal axis to engage said threaded shaft with at least one of said load transfer element and said pressure pad.

10. The tourniquet device of claim 1 wherein said collar restrictedly translates with said load transfer element via engagement with said threaded shaft as said spring element stores and releases energy in applying pressure to a patient.

11. The tourniquet device of claim 9, wherein said collar is configured to remain in position after pivoting said approximately 90 degrees from a stowed configuration to an active configuration such that said pivoting is not reversible.

12. The tourniquet device of claim 1 wherein said load transfer element engages with the exterior of said threaded shaft.

13. The tourniquet device of claim 1 wherein said load transfer element engages with the interior of said threaded shaft.

14. The tourniquet device of claim 1 further comprising at least one spool carriage configured to facilitate winding and unwinding of said spool.

15. The tourniquet device of claim 1 further comprising at least one spool carriage configured for mounting said at least one ratchet pawl, said at least one pawl release lever, or both.

16. The tourniquet device of claim 1 further comprising a pawl release bar engaged with said at least one pawl release lever and configured to facilitate movement of said at least one pawl release lever.

17. The tourniquet device of claim 1 further comprising a slack removal knob configured to facilitate winding said strap around said spool.

18. The tourniquet device of claim 1 further comprising a housing that contains at least said spool and said strap.

19. The tourniquet device of claim 1 wherein said collar and said load transfer element are merged into a single component.

20. A method for restricting flow of blood in a junctional location of a patient with a tourniquet device, said method comprising:
orienting said tourniquet device proximate to said junctional location and rotating a pressure pad to align with anatomy of the patient;
applying direct pressure over said junctional location through said tourniquet device;
activating a pawl release lever that enables a length of tourniquet strap to be unwound from a spool and a length of said tourniquet strap to be pulled out to wrap around the patient;
wrapping said tourniquet strap around the patient to form a loop by coupling the free end of said tourniquet strap to a base plate, said base plate also holding the fixed end of said tourniquet strap;
removing any slack in said tourniquet strap that is wrapped around the patient;
pivoting a tightening knob from a stowed position to an active position; and
rotating said tightening knob to increase pressure applied to the patient until bleeding has stopped or until a user feedback mechanism indicates that sufficient pressure has been applied.
